(19) United States
(12) Patent Application Publication
(10) Pub. No.: US 2025/0325730 A1
(43) Pub. Date: Oct. 23, 2025

(54) DUAL-PURPOSE GEL FOR ENDOMETRIOSIS MANAGEMENT AND CONTRACEPTION

(71) Applicants: EMPA EIDGENÖSSISCHE MATERIALPRÜFUNGS-UND FORSCHUNGSANSTALT, Duebendorf (CH); ETH ZÜRICH, Zürich (CH)

(72) Inventors: Alexandre Hermann Christos ANTHIS, Zürich (CH); Inge Katrin Herrmann, Siebnen-Wangen (CH); Samuel Kilchenmann, Zürich (CH); Thomas Rduch, St. Gallen (CH)

(73) Assignees: EMPA EIDGENÖSSISCHE MATERIALPRÜFUNGS-UND FORSCHUNGSANSTALT, Duebendorf (CH); ETH ZÜRICH, Zürich (CH)

(21) Appl. No.: 19/042,964

(22) Filed: Jan. 31, 2025

(30) Foreign Application Priority Data
Mar. 28, 2024 (EP) 24 167 147.8

(51) Int. Cl. A61L 27/52; A61L 27/16; C08L 33/26

(52) U.S. Cl. CPC A61L 27/52; A61L 27/16; C08L 33/26; A61L 2430/22; A61L 2430/36

(57) ABSTRACT

The present invention relates to a hydrogel that can be used to prevent or treat endometriosis and as mechanical contraception. Said hydrogel comprises a crosslinked network of at least
a) a first monomer unit selected from the group consisting of acrylamide-2-methylpropanesulfonic acid (AMPS), acrylic acid, sodium acrylate, polyethylene glycol acrylate, (acrylamidopropyl)trimethylammonium chloride, methacrylic acid, 2-(dimethylamino)ethyl acrylate, 2-aminoethyl acrylate hydrochloride and 2-acryloxyethyltrimethylammonium chloride or a mixture thereof,
b) a second monomer unit selected from the group consisting of N-2-hydroxyethyl acrylamide (NHEA), N-2-hydroxypropyl acrylamide (NHPA), acrylamide, N-isopropylacrylamide (NIPAm), hydroxyethyl methacrylate (HEMA), hydroxyethyl acrylate (HEA), methyl acrylate, butyl acrylate, ethyl hexyl acrylate, methacrylamide, and ethyl hexyl (meth)acrylate or a mixture thereof,
c) at least one crosslinker with dual functionality, having at least two crosslinking groups and at least one degradable moiety selected from the group consisting of a photo-labile group, a disulfide bond and a diselenide bond.

The present invention relates to a gel that can be used to prevent or treat endometriosis and as mechanical contraception.

The fallopian tubes are integral to female fertility, facilitating the transit of eggs and sperm and enabling fluid exchange with the uterus. Female sterilization, which involves tying off, sealing, or excising fallopian tubes, is the second most prevalent contraceptive strategy in the United States. By performing these procedures, the movement of eggs and access of sperm are obstructed, effectively preventing fertilization.

Although sterilization methods have a high adoption rate due to their effectiveness and the appeal of non-hormonal options, a notable proportion of women-exceeding 10%-regret this permanent decision. Regret after sterilization often stems from subsequent health issues, such as gynecological or menstrual disturbances, or the later desire to conceive.

While it is possible to surgically attempt to reverse the sterilization, the process is fraught with challenges, including significant expense and no assurance of restored fertility. Despite these potential drawbacks, permanent sterilization through disrupting fallopian tube function maintains its stature as a reliable contraceptive option, largely due to its proven track record in preventing unwanted pregnancies.

Hysteroscopic sterilization offers a less invasive option for permanent contraception, with the Essure® device as a notable example. This device, a micro-insert placed at the entrance of the fallopian tubes, promotes fibrosis, resulting in tube blockage. Its transcervical insertion allowed for a straightforward, efficient, and low-cost sterilization process, lasting about 15 minutes at a doctor's office without general anesthesia. Despite its initial popularity, Essure® was discontinued in 2018 due to significant post-procedural complications-over 32% of patients experienced adverse gynecological effects, and around 15% needed the device surgically removed.

While the early acceptance of Essure® showed the potential for non-surgical fallopian tube occlusion, the development of new materials continues. For instance, Femasys, Inc. is in the process of creating a biodegradable polymer liquid (FemBloc®) that is designed to be administered into the fallopian tubes using a catheter-based method. This substance is intended to induce scarring and blockage of the tubes within three months following the injection. Despite innovations, such approaches have seen some complications and remain challenged by the unresolved issue of safely reversing the sterilization should a woman wish to conceive again.

US20200352649 discloses methods for temporarily blocking a body passageway using a stimuli-responsive polymer that degrades upon exposure to certain stimuli, such as light. The process involves injecting substances into the lumen, which then form a polymer mass capable of effectively preventing the passage of materials through it.

US2020237388 details a system that delivers stimuli to an implantable occlusive device, which could be made from materials like hydrogels. This device is designed to provide reversible contraception by blocking the was deferens, fallopian tubes, or uterus, and can also be applied to occlude other body ducts or organs.

Endometriosis is a chronic gynecological condition characterized by the growth of tissue similar to the uterine lining in areas outside the uterus, especially within the peritoneal cavity. It affects roughly 10% of women in their reproductive years, leading to debilitating symptoms such as chronic pelvic pain, painful menstruation, pain during intercourse, and infertility. Despite the significant impact on the physical and mental health of those affected, a lack of public knowledge contributes to late diagnosis and therapies that are often only moderately successful.

Current treatments, including surgery to remove lesions, anti-inflammatory drugs, and hormonal therapies, show varied results and can result in adverse side effects and symptom recurrence within a few years for about half of the patients. The most established theory of endometriosis, implicates retrograde menstruation, where menstrual cells flow backward through the fallopian tubes, as a key mechanism.

While the exact cause of endometriosis is still not fully understood, interventions that prevent retrograde menstruation could significantly improve the quality of life for women with endometriosis.

The problem of the present invention is to provide a reversible technique for female reproductive tract management that can provide broad-spectrum benefits by effectively preventing pregnancy and addressing pelvic pain disorders such as endometriosis.

The problem is solved by a gel according to claim 1. Further preferred embodiments are subject of dependent claims 2 to 24.

The gel according to the present invention serves as a dual-function fallopian tube occlusive agent, blocking the movement of sperm, oocytes, and endometrial cells, thereby preventing both fertilization and the formation of endometrial plugs in the peritoneal cavity.

The gel comprises a crosslinked network of at least
a first monomer unit selected from the group consisting of acrylamide-2-methylpropanesulfonic acid (AMPS), acrylic acid, sodium acrylate, polyethylene glycol acrylate, (acrylamidopropyl) trimethylammonium chloride, methacrylic acid, 2-(dimethylamino)ethyl acrylate, 2-aminoethyl acrylate hydrochloride and 2-acryloxyethyltrimethylammonium chloride or a mixture thereof;
a second monomer unit selected from the group consisting of N-2-hydroxyethyl acrylamide (NHEA), N-2-hydroxypropyl acrylamide (NHPA), acrylamide, N-isopropylacrylamide (NIPAm), hydroxyethyl methacrylate (HEMA), hydroxyethyl acrylate (HEA), methyl acrylate, butyl acrylate, ethyl hexyl acrylate, methacrylamide, and ethyl hexyl (meth)acrylate or a mixture thereof;
at least one crosslinker with dual functionality, having at least two crosslinking groups and at least one degradable moiety, selected from the group consisting of a photo-labile group, a disulfide bond and a diselenide bond.

The gel according to the present invention marks a considerable advancement in the field of female contraception and disease management, particularly for those desiring a non-hormonal and reversible method of birth control, as well as for women suffering from abdominal endometriosis who are looking for non-pharmaceutical treatment alternatives. Unlike permanent procedures that block the fallopian tubes, gel according to the present invention offers a temporary solution that can be easily applied without surgery, for example through the working channel of a hysteroscope or a catheter. Furthermore, the gel according to the present invention remains exceptionally soft when in its fully expanded state, effectively blocking the fallopian tubes through functional occlusion without inducing fibrosis.

Using either a hysteroscope or a catheter, both minimally invasive medical tools, the gel is accurately positioned within the fallopian tubes. The procedure is designed to be straightforward and minimally invasive, providing a convenient option for women. Once inserted, the gel expands, filling the lumen of the fallopian tubes to create an effective physical barrier. This swollen state of the gel is integral to its function, as it provides a reliable and reversible blockade, which is both effective and biologically compatible with the body's tissues.

The practical implications of the gel's application are profound in that it prevents the passage of sperm cells, thereby averting fertilization and offering a safe approach to contraception. Additionally, for those dealing with abdominal endometriosis, the gel impedes the migration of endometrial cells through the fallopian tubes, thereby acting as a mechanical deterrent to the condition's progression and its associated symptoms. The gel thus stands as a purely mechanical intervention for two significant gynecological concerns.

The gel according to the present invention comprises two different types of monomer units. The first monomer unit is selected from the group consisting of acrylamide-2-methylpropanesulfonic acid (AMPS), acrylic acid, sodium acrylate, polyethylene glycol acrylate, (acrylamidopropyl) trimethylammonium chloride, methacrylic acid, 2-(dimethylamino)ethyl acrylate, 2-aminoethyl acrylate hydrochloride and 2-acryloxyethyltrimethylammonium chloride or a mixture thereof. Each of the mentioned monomer units includes at least one charged or water interacting group with significant hydrophilic characteristics, thereby enhancing the water absorption capacity of the resulting gel. Thus, the first monomer unit is responsible for a high swelling ratio, which is required for successful tissue compatible tubal blockage and subsequent efficient, stimuli-induced degradation. Within the context of the present invention, the term “water interacting group with significant hydrophilic characteristics” means chemical groups that demonstrate a strong affinity for water due to their ability to form hydrogen bonds or engage in electrostatic interactions with water molecules. Taking “polyethylene glycol acrylate” as an example, the water interacting group is the polyethylene glycol (PEG) segment. Most preferably, the first monomer unit carries a charge and is selected from the group consisting of acrylamide-2-methylpropanesulfonic acid (AMPS), acrylic acid, sodium acrylate and methacrylic acid. Owing to their charge, they engage in strong electrostatic interactions with water molecules, resulting in significant swelling.

The second monomer unit is selected from the group consisting of N-2-hydroxyethyl acrylamide (NHEA), N-2-hydroxypropyl acrylamide (NHPA), acrylamide, N-isopropylacrylamide (NIPAm), hydroxyethyl methacrylate (HEMA), hydroxyethyl acrylate (HEA), methyl acrylate, butyl acrylate, ethyl hexyl acrylate, methacrylamide, and ethyl hexyl (meth)acrylate or a mixture thereof and prevents excessive swelling and instability of fully hydrated gels while providing additional biocompatibility. Additionally, the presence of this group contributes to the improved structural integrity of the swollen gel when it is saturated with water. Most preferably, the second monomer unit is selected from the group consisting of N-2-hydroxyethyl acrylamide (NHEA), N-2-hydroxypropyl acrylamide (NHPA), acrylamide, N-isopropylacrylamide (NIPAm), hydroxyethyl methacrylate (HEMA), butyl acrylate and methyl acrylate, ideally N-2-hydroxyethyl acrylamide (NHEA) and N-2-hydroxypropyl acrylamide (NHPA).

The unique combination of these two types of different monomer units results in a gel that exhibits superior swelling properties. This gel effectively blocks the fallopian tubes, ensuring a reliable seal, while concurrently maintaining a pressure-free state to avoid discomfort for the patient.

Additionally, the gel according to the present invention comprises at least one crosslinker that bonds either the first monomer unit, the second monomer unit, or both, resulting in the formation of the gel. Said crosslinker has a dual functionality, having at least two crosslinking groups and at least one degradable moiety selected from the group consisting of a photo-labile group, a disulfide bond and a diselenide bond.

Crosslinking of the components usually begins with well-known initiators such as ammonium persulfate (APS) or tetramethylethylenediamine (TEMED). These initiators help in the formation of free radicals, which then interact with the first monomer units, the second monomer units, and the crosslinker present in the solution, leading to the polymerization process. This interaction facilitates the creation of covalent bonds between the monomers and the crosslinker, effectively forming a three-dimensional network.

Within the context of the present invention, the term “at least two crosslinking groups” refers to functional moieties present in the crosslinker molecule that are capable of forming covalent bonds with multiple monomer units within the polymer matrix. The interaction between these monomer units and the crosslinker initiates the formation of a covalent bond, facilitating the development of a three-dimensional polymer network. Preferably, the at least two crosslinking groups are selected from the group consisting of acrylamide, acrylate, methacrylate, vinyl and epoxy.

Furthermore, the crosslinker has at least one degradable moiety selected from the group consisting of a photo-labile group, a disulfide bond and a diselenide bond.

Within the context of the present invention, the term “photolabile moiety” refers to a group within the crosslinker that undergoes a chemical change, which involves the breaking of a covalent bond upon exposure to ultraviolet or visible light. Preferably, the photolabile moiety is selected from the group consisting of O-nitrobenzyl alcohols, O-nitrobenzyl esters, O-nitrobenzyl ethers, O-nitrobenzyl amides, 0-nitrobenzyl carbamates, coumarinyl esters, coumarinyl ethers, coumarinyl amides, coumarinyl carbamates, phenacyl esters, pyrenylmethyl esters, and benzoin ethers.

Preferred crosslinkers with a photolabile group are selected from the group consisting of PEG diacrylate (PEGDA), zinc dimethacrylate, zinc diacrylate, and PEGdiPDA. An especially preferred crosslinker with a photolabile group is PEGdiPDA of the general formula I

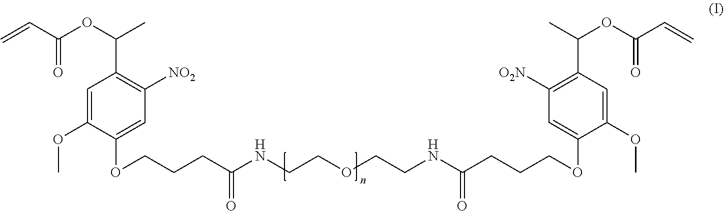

comprising two acrylate groups as crosslinking groups and two O-nitrobenzyl esters as photolabile groups.

In the context of the present invention “disulfide bond or diselenide bond” denotes a bond in the crosslinker that breaks when exposed to a thiol compound or free radical source. Preferably, such crosslinkers are selected from the group consisting of carboxybetaine disulfide cross-linker (CBX-SS), diselenide crosslinker and N,N′-bis(acryloyl)cystamine (BAC). An especially preferred crosslinker with disulfide bond is N, N′-Bis(acryloyl)cystamine of the general formula (II)

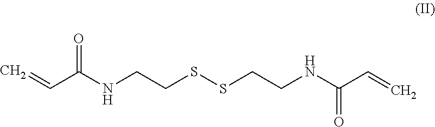

comprising two acrylate groups as crosslinking groups and central disulfide bond as degradable moiety.

The two degradation mechanisms ensure that the gels according to the present invention can break down upon exposure to either ultraviolet (UV) light, or a chalcogen-degrading agent (i.e. disulfide and diselenide bonds), facilitating removal within timeframes that are relevant to clinical applications. Degradation of the gel according to the present invention can be achieved for example by light irradiation (λ=365 nm) with a medical optical fiber (I0=40 mW/cm2) within less than 30 minutes. Likewise, gels comprising a thiol-labile moiety can be degraded upon exposure to disulfide reducing agents, such as glutathione (GSH). Such a disulfide or diselenide reducing agent can be applied by using a common fallopian tube perfusion system and results in degradation of the gel within 30 minutes (cGSH=200 mM). Thus, for both degradation mechanisms essentially complete gel degradation can be achieved.

In addition to the above-mentioned components, the gel according to the present invention may comprise additional other ingredients known to the skilled person.

Excellent results could be obtained with a gel comprising acrylamide-2-methylpropanesulfonic acid (AMPS) as first monomer unit, N-hydroxyethyl acrylamide (NHEA) as second monomer unit and PEGdiPDA or N, N′-bis(acryloyl)cystamine as crosslinker. It was shown that the presence of AMPS enabled the achievement of high swelling ratios, leading to an effective tubal blockage and promoting efficient degradation in response to specific stimuli. The presence of NHEA monomers in the gel formulation acts to mitigate issues of excessive swelling and instability once the gel is fully hydrated, while concurrently improving the biocompatibility of the system.

Within the context of the present invention, the term “gel” encompasses materials classified based on their water content as hydrogels, xerogels, or aerogels. Each type is defined by its unique water interaction and structural traits. The term “hydrogel” stands for a gel with a liquid content above 20% by weight. However, the hydrogel according to the present invention have the remarkable ability to absorb additional water. This capability is rooted in the unique composition of the two different monomer units. Preferably, the hydrogels according to the present invention have a relative swelling ratio of 2 to 20.

One aspect of the present invention relates to a hydrogel. As a hydrogel, the gel according to the present invention, when in its fully expanded state, exerts its full effect by effectively blocking the fallopian tubes through functional occlusion.

Preferably, the combined weight percentage of the resulting polymer network after polymerization and before ethanol assisted drying within the resulting hydrogel, ranges from 20 to 60%, with an even more preferable range being from 25% to 40%, indicating the total gel content. A concentration of less than 20% of the combined monomer units would lead to a mechanically inadequate implant, while a concentration exceeding 60% may cause an excessively high swelling rate which in return could damage the tissue area consisting of the implantation area.

A further embodiment relates to a xerogel or aerogel made from a hydrogel according to the present invention. Within the context of the present invention, the term “xerogel” stands for a hydrogel that has been dried to have a liquid content below 20% by weight, preferably of less than 5% weight, while the term aerogel stands for a hydrogel lyophilized via water sublimation or following supercritical drying. The term liquid stands for water, ethanol or a mixture thereof. Said xerogels or aerogels exhibit excellent storage stability and allow for the prediction of their swelling behavior to be accurate.

A further aspect of the present invention pertains to hydrogels or xerogels or aerogels prepared from the described hydrogels, shaped into tubes for easy insertion through a urinary catheter. To create such tubular hydrogels, a non-reactive, often fluoropolymer-based, tube is used as a mold. A mixture of the two different monomer units and the crosslinker is introduced into the tube. Following the addition of an initiator that triggers the chemical reaction, the compounds undergo a polymerization process within this tube. Following the initial processing, the hydrogel may then be subjected to an optional incubation period in ethanol, pure water, or a combination of ethanol and water, lasting anywhere from 1 to 4 hours. Subsequently, these liquids can be eliminated by placing the gels in a vacuum oven to dry, which leads to the formation of xerogels.

Another aspect of the present invention pertains to the application of the gels in the treatment and/or prevention of endometriosis and its clinical manifestations. As previously stated, the gels have been demonstrated to obstruct the passage of endometrial cells effectively, thanks to their compatibility with tissues and the resultant tubal blockage.

The gel of the invention can be administered before the onset of endometriosis to prevent its occurrence, immediately following the onset of endometriosis, or during the ongoing manifestation of endometriosis, whether the condition is acute or chronic.

The term “prevention” refers to the prevention or reduction of signs and symptoms associated with endometriosis in subjects who are at risk for developing endometriosis. In these subjects a predisposing factor may be retained, but the signs and/or symptoms of endometriosis do not occur or take significantly longer to develop. Further, it also includes the prevention of a further deterioration of the symptoms once endometriosis has occurred. In that respect, prevention denotes a reduction in the likelihood (chance) of the development and/or progression of the signs and/or symptoms of endometriosis. Thus, prevention relates to prophylactically treating endometriosis in that manner (such that, in contrast, therapeutically treating endometriosis mainly applies after endometriosis development). Furthermore, the gel according to the present invention functions as a contraceptive for a female individual. But if a woman decides to pursue pregnancy, the hydrogel can be conveniently removed without resulting in scars, and a new gel can be put in place after she has become pregnant. Thus, the gel with its dual function that treats endometriosis while also providing birth control, presents a significant benefit for women managing this condition. It offers the convenience of an all-in-one treatment, addressing both the painful symptoms of endometriosis and the need for reliable contraception.

One of the key advantages of the gel is its easy removability, which empowers women to plan for pregnancy when they feel ready, with the expectation of a quick return to fertility once the device is removed. This feature is particularly important for those concerned about their reproductive health, as endometriosis can impact fertility. Additionally, after childbirth, the simple reinsertion of a new gel enables the resumption of both endometriosis management and contraceptive protection, providing continuity of care.

Another aspect of the gel according to the present invention relates to its use as a contraceptive for female individuals. In addition to providing reliable protection against pregnancy, it allows for non-hormonal contraception. By choosing a non-hormonal and easily removable contraceptive option from the fallopian tubes, individuals can steer clear of the hormonal fluctuations associated with other forms of birth control like the pill, patch, or hormonal IUD. This means avoiding potential side effects such as mood swings, weight gain, and other systemic symptoms that some users experience with hormonal methods.

Additionally, the hydrogel according to the present invention can be smoothly extracted from the fallopian tubes, allowing for a rapid restoration of fertility. Unlike hormonal contraceptives that can take some time to wear off after discontinuation, the hydrogel can be removed with immediate effect on fertility. This is particularly appealing for users who wish to maintain control over their reproductive plans without enduring a waiting period for their natural fertility to return. The use of the gel according to the present invention is also advantageous since it allows for long-term family planning without the need for daily or even monthly maintenance. Once in place, it can provide continuous protection for years, minimizing the risk of human error, such as forgetting to take a pill or replace a patch.

A further aspect of the present invention relates to the introduction of the gel into a body lumen, preferably fallopian. This method involves
a) Inserting a catheter into the body lumen,
b) introducing the gel according to the present invention into the catheter, and
c) pushing said gel into the body lumen.

To prevent the gel from adhering, an encasement like a Teflon tube may be employed. The blockage of the body lumen can be monitored using ultrasound imaging. This method enables a swift insertion using tools that are readily accessible in any physician's office, and it can be performed without causing pain.

A further aspect of the present invention relates to the removal of the hydrogel according to the present invention.

In case of a hydrogel comprising a photolabile crosslinker, the method involves
a) inserting a medical instrument comprising a light source, for example a medical fiber that is coupled to an LED light source,
b) irradiating the hydrogel, for example by light irradiation (λ=365 nm) with an intensity level of I0=40 mW/cm² within less than 30 minutes.

Optionally, the body lumen can be flushed with fluid after the procedure is completed and also periodically during the treatment, facilitating the expedient clearance of any residual traces of the hydrogel.

In case of a hydrogel comprising a crosslinker with disulfide or diselenide bonds, the method involves
a) administering a solution containing disulfide or diselenide reducing agent into the body lumen. A possible disulfide or diselenide reducing agent is glutathione.

Optionally, the body lumen can be flushed with further fluid such as water or saline after the procedure is completed.

The invention is further illustrated by the following figures and examples.

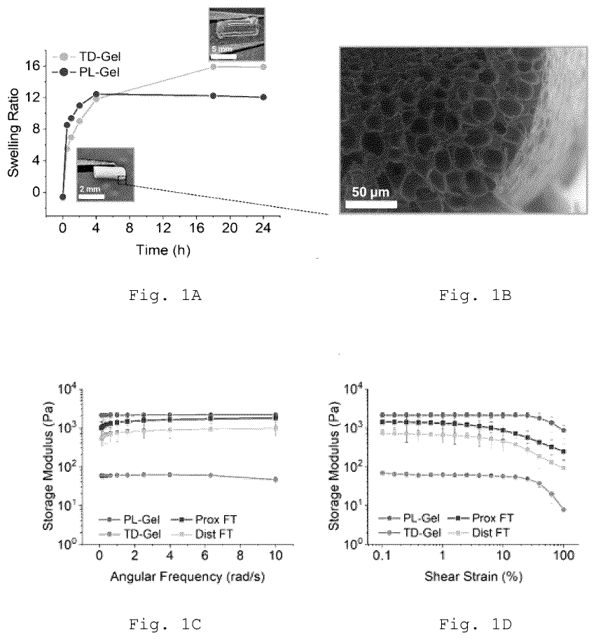

FIG. 1a shows the swelling behavior of photolabile (PL) and thiol-labile (TD) gels in simulated oviduct fluid (SOF).

FIG. 1b shows a scanning electron micrograph of an as-applied TD-Gel.

FIG. 1c shows storage moduli of PL and TD gels as a function of frequency.

FIG. 1d shows the shear strain.

FIG. 1e shows the change in storage modulus over time upon light exposure indicating gel degradation.

FIG. 1f shows a photograph of medical optical fiber degrading hydrogel-blocked, porcine fallopian tube in a background of simulated fallopian tube fluid. Data displayed as mean±standard deviation. Measurements were performed in triplicates.

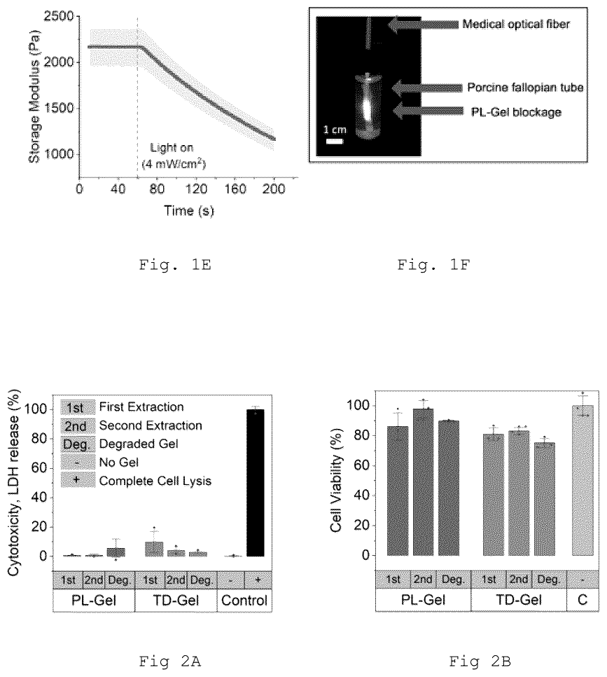

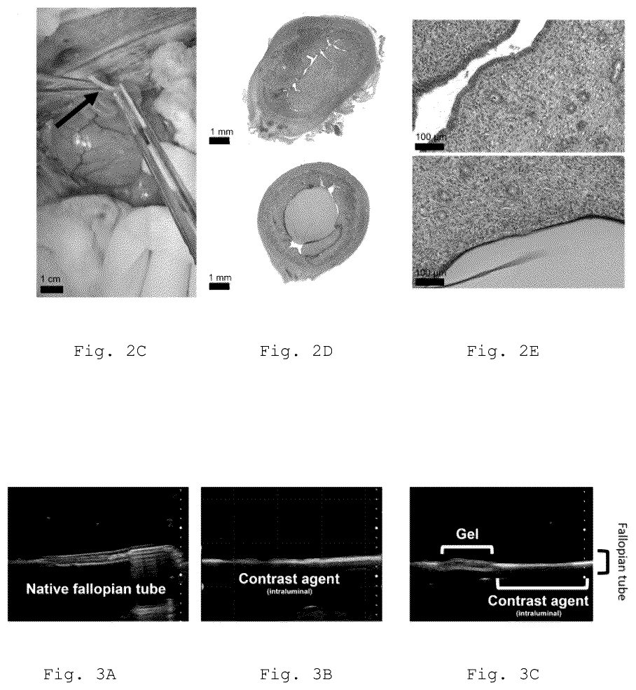

FIG. 2a shows lactate dehydrogenase (LDH) release cytotoxicity assay of human fibroblasts cells cultured in cell medium subjected to different states of PL and TD hydrogel particles. Cell culture medium and medium containing 0.1% TritonX were used as negative (grey) and positive (black) controls, respectively 24 h.

FIG. 2b shows viability of human fibroblasts after 24 h of incubation with hydrogel conditioned media.

FIG. 2c shows surgical placement of hydrogel into pig fallopian tubes in vivo for long term biocompatibility assessment.

FIG. 2d-e show H&E staining of porcine oviduct sections (isthmic region). (top) patent (non-blocked) oviduct. (bottom) oviduct blocked with TD-Gel for 3 weeks in vivo.

FIG. 3a-c show monitoring of the effective blocking by the hydrogel implants under ultrasound using the contrast agent ExEm foam. Ultrasound image of a native fallopian tube, the unblocked fallopian tube with intraluminal ExEm foam injection, and the blocked fallopian tube with ExEm foam injection is shown.

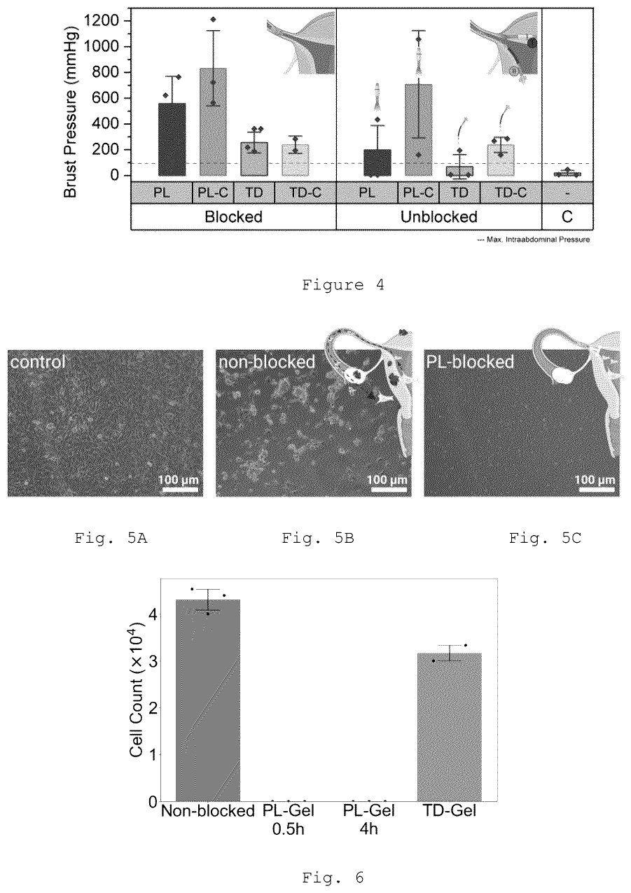

FIG. 4 shows burst pressure data of fallopian tube sections using simulated fallopian tube fluid. Left: After blockage with PL- and TD-Gels. Right: Fallopian tube samples after blockage and subsequent reversal using light (turquoise) or thiols (salmon) and patent fallopian tube sections (black). Maximal abdominal pressure reported in literature is indicated by the dotted line.

FIG. 5a-c shows the assessment of endometrial cell passage through non-blocked and PL-blocked fallopian tubes. Control cell culture of endometrial cells (12Z) shown on the left.

FIG. 6 shows the number of endometrial cells which passed the fallopian tube during 30 min (0.5 h) of continuous back and forth flow of cell medium (1 mio cells in 4 mL cell medium). Non-blocked tube, 40,000 cell passed through non blocked fallopian tubes. No cells passed through fallopian tubes blocked with our photolabile gel whether the experiment was conducted for 30 min or 4 h and approx. 30,000 cells passed through the fallopian tube sections blocked with Thiol-labile gel (however, pressure led to burst of gel out of tube section).

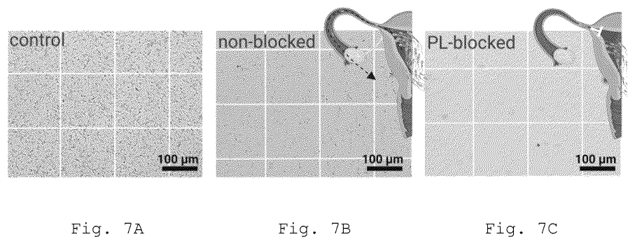

FIG. 7a-c show assessment of live porcine sperm passage through non-blocked and PL-blocked fallopian tubes. All experiments were performed at least three times using independent samples (N=3).

### EXPERIMENTS

### Chemicals and Materials

All chemicals were purchased from Sigma-Aldrich except Formalin (ROTI® Histofix 4%, ROTH) and PEGdiPDA crosslinker. The PEGdiPDA molecule was synthesized following literature protocols (Kloxin, A. M.; Tibbitt, M. W.; Anseth, K. S. Synthesis of Photodegradable Hydrogels as Dynamically Tunable Cell Culture Platforms. Nat Protoc 2010, 5 (12), 1867-1887. https://doi.org/10.1038/nprot.2010.139). PEGdiPDA crosslinker and solutions containing the photolabile molecule were always stored in dark and protected from light during handling. N-(2-Hydroxyethyl) acrylamide (NHEA) monomer solution and poly(ethylene glycol) diacrylate Mn 4,000 Da (PEGdiacrylate) both contained MEHQ inhibitor and were purified by running them through a 1 cm column of basic alumina oxide (Brockmann Grade I). As PEGdiacrylate was provided in solid form, dissolution in 98% ethanol prior to purification was necessary. Later, ethanol was removed from the PEGdiacrylate filtrate using a rotary evaporator. Clean NHEA was stored at 4° C. and clean PEGdiacrylate at −20° C. Teflon tubes used to obtain tube shaped hydrogels were bought at ETHZ HCl Shop, 3D printing material (PETG) was purchased from Prusa. The plastic mold used for the uterus model was purchased at a local convenience store. Fresh fallopian tubes were provided by a local slaughterhouse (SBZ Schlachtbetrieb Zuirich AG). Tissue samples were either used fresh (Histology) or stored at −20° C. in double sealed freezer bags. Samples were always thawed immediately before use and never refrozen. Endometrial cells (12Z) and materials used for cell culture were purchased from Abm (Applied Biological Materials Inc., USA).

### Simulated Oviduct Fluid Preparation

The composition of simulated oviduct fluid was adapted from descriptions biological fluid descriptions of J. Aguilar et al The uterine tubal fluid: secretion, composition and biological effects. (Periodikos, http://www.animal-reproduction.org/article/5b5a6085f7783717068b47ed (accessed 2023-09-24)). More specifically a 1 L stock of simulated oviduct fluid (SOF) precursor was prepared by dissolving 5.174 g NaCl, 0.91 g KCl, 0.24 g CaCl₂ dihydrate, 0.207 g MgCl₂ hexahydrate, 1.747 g NaSO₄ and 2.329 g Na₂HPO₄ heptahydrate in 1 L Milli-Q water. The precursor solution was stored at RT and stirred for 10 min on a stirring plate (500 rpm) before use. To prepare 0.2 L of final SOF stock, 19 mg Glucose, 192.2 mg Lactate, 3.5 mg Pyruvate and 1.4 g Albumin were dissolved in 0.2 L of SOF precursor solution. pH of final SOF was adjusted to 7.2 using NaOH or HCl. pH adjusted SOF was stored at 4° C. for no more than 1 week before use.

### Gel Preparation

Prior to preparing gel master mixes (MM), a stock solution of 2 wt % N, N′-Methylenebisacrylamide (mBAA) was made by dissolution of 1 g (6.5 mmol) mBAA in 49 mL Milli-Q water. Additionally, a 2 wt % N, N′-Bis(acryloyl)cystamine (BAC) crosslinker stock was prepared by dissolving 40 mg (153.6 μmol) BAC in 980 μL Milli-Q water and 980 μL Isopropanol. Both crosslinker stocks were sonicated for 10 min and stored at 0-4° C. Furthermore, an Ammonium Persulfate (APS) stock was prepared by dissolving 60 mg APS in 2 mL Milli-Q water. The APS stock was vortexed and stored at 0-4° C.

To prepare the photolabile (PL) gel MM and its PEGdiacrylate control (PL-C), a 40 wt % monomer mix of 2-acrylamido-2-methyl-1-propanesulfonic acid (AMPS) and clean, inhibitor removed, N-(2-hydroxyethyl) acrylamide (NHEA) was made. For a 2 mL mix, 640 μL AMPS, 50 μL NHEA, 530.6 μL Milli-Q and 2.42 μL TEMED were added to a 15 mL falcon tube and vortexed for approximately 1 min. Subsequently, 33.35 mg (16.7 μmol) of crosslinker (PEGdiPDA or PEGdiacrylate) was added to the mix and vortexed once more. To prepare the thiol-labile (TD) MM, a final 25 wt % AMPS/NHEA monomer mix was made. For a 2 mL mix, 800 μL AMPS (50 wt % in Milli-Q), 100 μL NHEA (pure, inhibitor free), 1063.5 μL Milli-Q and 2.42 μL TEMED were added in a 15 mL falcon tube and vortexed for 1 min. After mixing, 34.02 μL of 2 wt % BAC stock solution was added to the MM. An mBAA control (TD-C) MM was prepared similar to the TD-MM by replacing the 34.02 μL of 2 wt % BAC with 20.15 μL 2 wt % mBAA and 13.9 μL Milli-Q water. All MM were frozen in 100 μL aliquots (−20° C.) and thawed only once, immediately prior to use.

To obtain tubes shaped gels, a long Teflon tube (ID=1.14 mm) was cut into ˜1.5 cm long sections. A 100 μL MM aliquot was thawed, vortexed and 7.2 μL APS (30 mg/mL) was added to initiate polymerization (independent of MM type). After adding APS, gel solution was vortexed shortly (˜10 s) and injected into short Teflon tube sections (10 μL per Teflon tube section) using a 10 μL pipette. Gels were polymerized within the Teflon tubes for 15 min at 60° C. and incubated in 98% ethanol overnight at room temperature (RT). The following morning, gels were removed gently from tubing with a blunt syringe needle (20 G) and incubated in ethanol for two additional hours. Next, ethanol was removed, and gels were dried in a vacuum oven (10 mbar, 40° C.) over night. Dry gels were stored in tightly closed glass vials at room temperature.

These as-prepared gel articles were loaded into a catheter and inserted into the fallopian tube (ex vivo) through the working channel of a hysteroscope. The gel articles were pushed through the catheter and placed at the proximal part of both fallopian tubes (hysteroscopic insertion). Upon tissue contact, the gel article immediately started to swell, increasing in size to fill the fallopian tube. Within a few hours, the gel reached its swelling plateau, effectively blocking the fallopian tube and preventing sperm and endometrial cells from passing (tubal occlusion). At this point, if reversal of tubal blockage was desired, the gel could be degraded using light (PL-Gel) or thiol-containing fluids (TD-Gel). As described above, both removal methods can be applied easily in a minimally invasive procedure through the working channel of a hysteroscope resulting in refertilization.

### Swelling Experiments

For swelling experiments, samples of all four gel types (PL, PL-C, TD and TD-C) were prepared in triplicates. Gels were made similar to the tube-shaped gels described above. However, instead of polymerizing 10 μL of gel solution within Teflon tubes, 50 μL droplets were polymerized on Teflon plates. Also, prior to the drying process, each gel was transferred into a separate glass vial. The weight of each vial was measured with and without a gel and the “as-prepared” initial hydrogel mass Mᵢ was calculated.

$${M}_{i} = {M}_{Vial + Gel} - {M}_{Vial}$$

After the drying process, 5 mL of SOF was added to each vial and the samples were incubated at 37° C. on a lab shaker (IKA®KS 130 basic, 80 rpm). At different timepoints (0, 30 min, 1 h, 2 h, 4 h, 18 h, 24 h), remaining SOF was removed with a 20 G Sterican Syringe Needle and residual gel mass was calculated as described above. Relative swelling ratios were calculated as follows:

$${S}_{R} = \frac{{M}_{Swollen} - {M}_{i}}{{M}_{i}}$$

It is notable that for timepoint 0 min the M_{swollen} corresponds to the mass of the lyophilised gel with Mᵢ being the mass of the gel directly after radical polymerization.

### Gel Stability in Human Peritoneal Fluid.

Human peritoneal fluid was obtained during laparoscopic surgery on a woman in her childbearing years at Kantonsspital St. Gallen (KSSG). A laparoscopic suction device was used to remove approximately 14 mL of peritoneal fluid from the posterior cul-de-sac. Collected fluid was stored at −20° C. for less than one month. A general consent form was signed by the patient.

To tests gel stability in human peritoneal fluid, 2 mm long, as-applied hydrogels were immersed in in 2 mL of human peritoneal fluid. Gels were swollen for 24 h at 37° C., under slight shaking (80 rpm). After 24 h, remaining fluid was removed carefully, and gel stability was assessed qualitatively.

### Characterization of Stimuli-Removable Gel Systems

In situ swelling capacity and swelling kinetics for PL- and TD-Gels were estimated by full immersion of hydrogels in simulated oviduct fluid (SOF) ex situ (FIG. 1a). Hydrogel weight was measured at different time points and swelling ratios were calculated. The swelling plateau of stimuli-removable hydrogels was reached within 4-6 hours, resulting in a final swelling ratio of 12 (PL-Gel) and 16 (TD-Gel). The higher swelling equilibria of TD-Gels is attributed to its lower polymer weight fraction (25 wt % vs. 40 wt %) and lower crosslinker concentration (1.31 mM vs. 8.36 mM) compared to PL-Gels. However, since measurements were taken at full immersion in fluid (50 μL gel in 5 mL SOF) and without any compressive forces acting on the gel, a slower in situ swelling rate is expected as human tubal fluid production is limited and strongly dependent on the day of menstrual cycle. Typical daily fluid production ranges from 0.3 mL to 9.6 mL with its peak 1-2 days before ovulation. Therefore, implantation a few days prior to ovulation is recommended to ensure high swelling rates and prevent early dislocation of hydrogels. As-applied hydrogels were swollen in human peritoneal fluid for 24 hours, to assess the stability of PL and TD-Gels in relevant human biological fluid. Stability of fully hydrated hydrogels was quantitatively assessed by visual analysis and observed to be stable over the complete observation period (21 days). Long-term exposure to simulated oviduct fluid showed that the implants remained mechanically intact and stable for more than 6 months, indicating potential for long-term implantation. To assess as-applied hydrogel implant characteristics, scanning electron mi-crographs of a dry TD-Gel cross-section was recorded (FIG. 1b). A porous network was observed without apparent differences between different regions of the implant (center or periphery), which is ideal ensuring uniform hydrogel swelling.

### Rheology

Rheological measurements were used to characterize viscoelastic properties and degradation kinetics of swollen hydrogels. Three sets of experiments were conducted. First, polymerization times and storage moduli of as-prepared hydrogels were assessed by in-situ polymerization of PL and TD Gel master mixes (T=60° C.). Second, storage moduli of swollen gels (PL and TD) and porcine FT tissue samples were measured by rheology. To fit the rheometer geometry (d=8 mm, sandblasted), oviduct samples were cut longitudinally, flattened, and punched through with an 8 mm disposable biopsy punch (KAI® Medical). Gel samples were polymerized in (3 mm and 4 mm) silicone molds (McMaster-CARR Duro Red), dried, swollen and embedded in 4 wt % Agarose in PBS. Embedded gels were cut into 300 μm slices using a Compresstome® (VF-310-0Z, Precisionary). Hydrogel and tissue samples were placed on the rheometer (Anton Paar MCR 502) and kept wet with PBS during measurements. To determine degradation kinetics of PL-Gels, 300 μm PL-Gel samples were irradiated with UV light (λ=365 nm, I₀=9.6 mW/cm²) using a liquid light quid coupled to an LED light source (ThorLabs DC4104—4-Channel LED Driver). Photodegradation first-order rate constant was calculated by normalizing storage modulus (G′) data and performing linear regression on a data subset (0.5≤G′/G′₀<1). FT rheological experiments were conducted in triplicates. All other measurements were done once for each experimental group.

Storage moduli of porcine fallopian tubes were approximately two times lower as fully swollen PL-Gels however more than ten times higher than TD-Gels (FIGS. 1c, 1d). As expected, based on polymer concentration and hydrogel swelling, fully hydrated TD-Gels were softer than PL-Gels. As both hydrogels were of a similar modulus compared to fallopian tube tissue, significant distortion of the fallopian tube due to excessive hydrogel swelling is unlikely. Further rheological measurements were performed to quantify degradation kinetics of the swollen PL-Gel upon light exposure. The first-order effective degradation rate constant was calculated (keff/I0=24 cm2 mW-1 s-1) and indicates a higher rate of degradation for swollen PEG-diPDA crosslinked hydrogels compared to degradation rates reported for non-swollen PEG-based photolabile hydrogels (keff/I0=5-10 cm2 mW-1 s-1).40,41 This is expected, since after swell-ing of the PL-Gels the photoactive crosslinker is diluted compared to its non-swollen state leading to less light attenuation and fewer crosslinks per unit volume, suggesting a relationship be-tween swelling behavior and degradation kinetics. These faster degradation kinetics are advantageous for the desired application and can afford full hydrogel degradation using a medical optical fiber setup (FIG. 1e). Using the optical fiber setup and UV exposure of 30 min for full degradation, we did not observe any damage to host tissue. While the light irradiation conditions utilized in this work are considered safe, the fallopian tube tissue integrity and fertility is to be further monitored carefully prior to clinical use. Degradation by irradiation with blue light (λ=405 nm) may be explored as an alternative as the nitrobenzyl groups of the PEGdiPDA are known to degrade at this wavelength.40 Overall, the conditions for degradation utilized here were selected to minimize the chance for damage to the host. Note also, that the glutathione degradable option presented here, may be used either alone or in combination with light, thus further reducing the irradiation dose. Compared to the permanent fallopian tube scarring introduced by alternative sterilization methods (REF), the above conditions likely offer relevant benefits with regard to refertilization potential (REF). Overall, both the PL- and TD-Gels have high swelling ratios that can lead to fallopian tube occlusion with mechanical properties that are not anticipated to lead to significant distortion of the fallopian tube and can readily be removed using a tissue-compatible trigger.

### Surgical Application Model

### Uterus Model

To create a negative model of the uterine cavity and the fallopian tubes, a 22 wt % acrylamide/gelatine mix was polymerized around a 3D printed model of the uterus. More precisely, an anatomical model of the human uterus (with fallopian tubes) was downloaded from sketchfab.com and modified using Tinkercad. Fallopian tubes were elongated, and the entire model was scaled to fit the size of a human uterine cavity. The modified model, together with a 10 cm long cylinder (d=2.5 cm) was printed with a PrusaMini 3D Printer using Prusament PETG filament. The cylinder was attached to the cervical part of the model and the inner wall of a 2 L plastic mold. Hot glue was allowed to harden overnight.

To prepare the 22 wt % acrylamide/gelatine mix, 40 g of Gelatine powder was dissolved in 1.56 L Milli-Q water by repetitive shaking and heating in a 60° C. preheated oven. Next, 400 g of Acrylamide was added to the mix under continuous stirring and heating (500 rpm, 60° C.). Once all gelatine and acrylamide were dissolved, 21.6 mL of 2 wt % mBAA Stock and 3.2 mL TEMED were added to the mix. Also, a 50 mL stock of Ammonium Persulfate (APS) solution was prepared by dissolving 3 g of APS in 50 mL Milli-Q.

The Tupperware was filled layer by layer with 200 mL of Aam/Gelatine mix. For each layer, 4.48 mL APS (60 mg/mL) was added drop by drop to 200 mL gel mix. After initiating polymerization, the gel was allowed to polymerize and cool down (at RT) for at least 30 min to avoid melting of the plastic mold and the 3D model. Final Aam/Gelatine model was stored at 0-4° C.

The uterus model was examined by a gynaecologist at the medical training center in St. Gallen (Ostschweizer Schulungs- und Trainingszentrum, KSSG). The Aam/Gelatine model was placed in a water bath and a hysteroscope (Karl Storz, 26120BA, 26153BIK, 26153BOK) was inserted through the cervix into the uterine cavity of the model. Images and videos were taken using the Tele Pack X LED Monitor (Karl Storz, TP 100). Gel insertion was simulated using a urinary catheter (Cook Medical, G14430) and a small Teflon tube (OD=0.8 mm).

### Ultrasound

Ultrasound images of our model were taken using a linear scanner (Clarius, Model L7HD) coupled to a smartphone using the Clarius Ultrasound App. Imaging was performed on the Aam/Gelatine model with patent and occluded fallopian tubes while immersed in a water bath.

### Gel Cytotoxicity and In Vivo Cytocompatibility

Perfused cell medium experiments were performed as described by Anthis et al. using CytoTox 96 Non-Radioactive Cytotoxicity Assay (Promega). Normal Human Dermal Fibroblasts (NHDFs), a non-cancerous human skin fibroblast cell line, was cultured under standard culture conditions at 37° C. with (5%) CO2. Dulbecco's Modified Eagle's Medium—high glucose (DMEM), supplemented with 10% Fetal calf serum, 1% L-Glutamine 1% Penicillin-Streptomycin-Neomycin Solution was used as full growth medium. Dried PL- and TD-Gel samples (20 μL) were swollen in 2 mL of full growth medium within (15 mL) falcon tubes at 37° C. After 24 h, media was removed (1st Extraction) and fresh media (2 mL) was added for a second incubation period of 24 h (2nd Extraction). A second set of samples, used to investigate cytotoxicity of degradation products, were swollen in 2 mL Dulbecco's Phosphate-buffered saline (PBS, D8537). After 24 h incubation, PBS was not removed, and swollen gels were degraded within the falcon tubes (Deg.). PL-Gel samples were irradiated with UV light (A=365 nm) for 30 min and TD-Gel samples were incubated at 80° C. for 24 h to break disulfide bonds. Extracted media and PBS of each experimental group was collected in (15 mL) falcon tubes, immediately frozen in liquid nitrogen and stored at −20° C. for less than one week.

4000 NHDF cells were seeded in (100 μL) full growth medium and allowed to attach for at least 24 h in a 96 well plate. 100 μL of extracted cell media or PBS (containing gel degradation products) was added to the cells (total volume 200 μL). Also, 100 μL of fresh PBS (negative control) and 100 μL full growth media containing 10 μL of 10× Lysis Solution (positive control) was added to the cells. The plate was then incubated under standard culture conditions for 24 h. Cell viability was assessed through an LDH cytotoxicity assay using CytoTox 96 Cytotoxicity assay (#G1780, Promega, Dubendorf, Switzerland). Three independent experiments were executed per experimental group.

An in vivo proof of feasibility study was performed using a piglet model. The study was conducted under the supervision of the Commission of Work with Experimental Animals at the Medical Faculty of Pilsen, Charles University, certified under project ID: MSMT-21623/2023-2, and was monitored by the Ministry of Agriculture of the Czech Republic. All procedures strictly adhered to the laws of the Czech Republic, which align with the regulations of the European Union. We conducted the proof-of-concept experiments in two pigs, and we present data from one representative pig. The subjects were healthy female Prestice black-pied pigs, 14 weeks old, weighing 30-40 kg, and they received intramuscular premedication with ketamine (Narkamon 100 mg mL-1, BioVeta a.s. Ivanovice na Hane, Czech Republic) and azaperone (Stresnil 40 mg mL-1, Elanco AH, Prague, Czech Republic). General anesthesia was initiated and maintained using propofol MCT/LCT (Propofol 2% MCT/LCT Fresenius Medical Care a.s.). Nalbuphin (Nalbuphin, Torrex Chiesi CZ s.r.o., Prague, Czech Republic) was used for analgesia. Midline incision laparotomy was performed to enter the abdominal cavity. The hydrogel implants were surgically implanted into the oviducts of two female piglets. The abdominal wall was closed by polydioxanone monofilament 1 suture (PDS II, 1, Ethicon, Johnson & Johnson, NJ) and the pigs were left to wake up and monitored closely for 21 days with ad libitium access to food and water. After 21 days, the piglets were anaesthetized and then immediately sacrificed using a cardioplegic solution and tissue samples were harvested and fixed in formalin. The entirety of the experiment was documented via photography.

### Burst Pressure

### Sample Preparation

Frozen fallopian tubes were thawed for 30 min in lukewarm water before use. First, the fallopian tubes and the uterotubal junction were identified. Soft tissue surrounding the fallopian tubes was dissected using surgical scissors. Residual parts of the uterine horn were removed together with the uterotubal junction and the proximal part (Isthmus) of the oviduct was cut into three (˜3 cm long) sections (s1, s2, s3). Blunt (22 G) syringe needles were carefully inserted into the tube sections and placed into a (15 mL) falcon tube filled with 5 mL of Formalin (ROTI® Histofix 4%, ROTH). Tissue sections were incubated in Formalin at room temperature (RT) for at least 24 h. After fixation, oviduct sections were removed, washed with PBS, and transferred into a different (15 mL) falcon tube containing 10 mL of PBS. Syringe needles were removed after 24 h incubation in PBS, oviduct sections were flushed with PBS and transferred in falcon tubes containing fresh (10 mL) PBS. After an additional incubation period of 4 h, fallopian tube sections were flushed again with PBS and as-applied hydrogels were inserted to block the tubes. More precisely, tube shaped gel samples were cut into 2 mm long sections with a scalpel and carefully inserted into ˜2 cm long Teflon tubes. The Teflon tube sections were then inserted approximately 1 cm deep into the fallopian tube and the gel was pushed into the FT using a (22 G) blunt syringe needle. For each gel type (PL, PL-C, TD and PT-C), tissue samples from the same fallopian tube were used (e.g., s1, s2 and s3 of FT 1 were all blocked with PL gels). After gel insertion, oviduct sections were incubated (at 37° C.) for 24 h, in 10 mL fresh SOF.

### Fallopian Tube Occlusion and Re-Fertilization

Constant physiological fluid movement and physiological pressures within the fallopian tubes have the potential to impact the long-term stability of the hydrogel's placement. The effective blocking of the tube was therefore further confirmed by ultrasound imaging using an ultrasound contrast agent (ExEm foam). Effective blocking of the contrast agent by the implant was observed by ultrasound (FIG. 3a, 3b, 3c). Functional hydrogel blockage and stimuli-responsive removal was further assessed using burst pressure measurements on blocked, unblocked, and control porcine fallopian tube sections (FIG. 4). For the expulsion of fully swollen hydrogels placed within the fallopian tube, average pressures of 558 mmHg (PL-Gels) and 255 mmHg (TD-Gels) were necessary. This significantly exceeds the highest measured intra-abdominal pressure of 95 mmHg as well as normal physiological pressures of 50 mmHg observed in the fallopian tubes. Burst pressure of in situ swollen PL-Gels decreased to 198 mmHg upon exposure to 30 minutes of light irradiation (λ=365 nm, I₀=40 mW/cm²) with an optical medical fibre. Burst pressure of TD-Gels decreased to 67 mmHg upon exposure to a 0.2M glutathione solution (dissolved in PBS, pH adjusted to 7.2) and continuous flow for 30 minutes. In TD-unblocked samples, measured pressures were very similar to the non-blocked control suggesting full degradation of two of the TD-Gels. The burst pressure data indicates that PL and TD-Gels are both valid systems for the blockage of fallopian tubes as they exceed maximal intra-abdominal pressures measured. However, even though significant decreases in the burst pressure were observed upon hydrogel exposure to stimuli, complete degradation of the hydrogels was only achieved for the TD-Gels and in not for the PL-Gels. In fallopian tubes blocked with non-stimuli responsive hydrogels (PL-C and TD-C), no significant difference between blocked and unblocked tubes is seen after exposure to degradation stimulus (FIG. 4). These measurements confirm that the observed significant decreases in burst pressure after light or reducing agent exposure are indeed related to the breaking of crosslinks in the macromolecular network. Once these crosslink are reduced to under 50%, the mechanical integrity of the gels is no longer given, and the degradation products are washed out from the fallopian tubes by physiological fluid circulating. Based on the cytocompatibility assessments performed, no adverse effects are to be expected and the degradation products are eventually excreted transvaginally.

### Endometriosis Model

As a final proof-of-concept, an in vitro model was developed to assess if the hydrogel could prevent retrograde menstruation and live sperm passage. Endometrial cells were flown through either a non-blocked or PL-Gel blocked fallopian tube using a setup similar to that used for burst pressure measurements. The passage of endometrial cells through the obstructed fallopian tubes were assessed. First, the exposure of lesion-forming endometrial cells to the flow systems cyclical increases and decreases in pressure (Δp=50 60 mmHg, freq. ˜1.6 cycles/min) was determined to not cause significant cell damage in the tested timeframe (30 min) compared to static control. After ascertaining that the setup did not cause cell damage, cells were passed through non-blocked and blocked fallopian tubes. Excitingly, the placement of the PL-Gel within the fallopian tube led to no cells being able to migrate through the tube (n=3), while approximately 40,000 out of 1 million available cells were able to pass through the non-blocked fallopian tube (n=3) over a period of 30 minutes (FIGS. 5A, 5B and 5C and FIG. 6). Microscopy images were utilized to qualitatively confirm the lesion forming nature of the endometrial cells. In addition, when the perfusion time was extended to 4 hours (this time using fixed cells to avoid cell lysis), no endometrial cells were found to pass through the fallopian tube. These analyses again confirmed that no cells passed through the occluded tube and no lesions were formed. Overall, the endometrial cell blocking proof-of-concept shown here demonstrates the potential utility of the developed stimuli responsive hydrogel system as a treatment option for endometriosis. Additionally, using boar sperm from a fertilization supplier, we show that no sperm cells were able to pass through the blocked fallopian tube even after repeated cycling (FIGS. 19A, 19B and 19C), while a significant number of sperm cells could pass through the non-blocked tube (1.85±1.36 million cells, n=3) from initially 19.6 million live sperm cells. Taken together, these experiments show that neither sperm nor endometrial cells are able to pass through fallopian tubes blocked with hydro-gel implants. How long-term blocking affects the host, and its fertility is yet to be investigated in comprehensive long-term studies, however, upon completion such investigations may help shine light on potential effects on menstrual cycle.

### Light Degradation

A 600 μm medical fiber (NA=0.37, SMA905/FC connector, Wuhan Medfibers Technology) was coupled to an LED light source (FC1-LED-365A, Prizmatix). Output power of the LED light source could be manually controlled. Peak light intensities of roughly 40 mW/cm² were measured at the tip of the medical fiber using a laser power & energy meter (SOLO PE, S/N 154961). The medical fiber coupled to the LED light source was used at maximal power to degrade photolabile hydrogels within FT segments. More specifically, the tip of the medical fiber (protective silicone removed) was inserted into a fixed and blocked FT section until slight resistance of the gel was felt. The LED light source was turned on and set to its maximal power. Gels were irradiated for a total of 30 min. After 10 min, 20 min and 25 min irradiation, the fiber was removed, and the FT section was flushed very carefully with (2 mL) SOF using a small (23 G) blunt syringe needle. Following light irradiation, samples were incubated again in SOF for 24 h at 37° C. FT sections blocked with PL and PL-C gels and irradiated with light were referred to as PL and PL-C unblocked FT samples.

### Thiol Degradation

To degrade TD-Gels within fallopian tube segments, a closed loop perfusion system was built. More precisely, a 3D printed hollow cylinder (PETG, ID=45 mm, OD=55 mm) was connected to a submersible mini water pump (DC 3-6V, flow rate ˜800 mL/min) using silicone tubing (ID=3 mm). The cylinder was divided into 4 compartments and the cylinder bottom (˜1 cm) filled with steel wool to avoid blockage of the system. Fixed FT segments blocked with TD- or TD-C-Gels were placed into each compartment. The perfusion system was filled with 100 mL 0.2 M Glutathione (GSH) solution (preheated to 37° C.). The GSH solution was prepared by dissolving 6.146 g L-Glutathione reduced in 50 mL PBS, adjusting the pH to 7.2 using (˜25 mL) 1M NaOH and adding additional PBS until a total volume of 100 mL was reached. The entire system was placed into a water bath (37° C.) and the pump was turned on. Following 30 min of continuous perfusion with GSH solution, samples were removed and incubated in fresh SOF for 24 h at 37° C. FT sections blocked with TD and TD-C gels perfused with GSH solution were referred to as TD and TD-C unblocked FT samples.

### Burst Pressure Measurements

The 50 mL syringe was filled with 10-20 mL fresh SOF and mounted on the syringe pump. The pumps flow rate was set to 1 mL/min and the silicone tubing system was attached to the syringe. The tubing was filled with SOF by tuning on the pump for approximately 30 seconds. Once all air was removed from the system, the three-way valve was closed. Next, the fallopian tube segments were attached to the (20 G) blunt syringe needle with a haemostat. For continuous pressure measurement, the sensor was connected to a computer and pressure values were monitored using a Sensor Evaluation Kit (Honeywell, SEK002 Version 1.1). Before attaching the sensor to the silicone tubing, pressure input was set to zero (Auto Zero). Once the sensor was attached, the pressure recording was started, and the syringe pump was turned on. The measurement was stopped once a significant drop in pressure was detected or no change in pressure occurred within 20 seconds. Three independent burst pressure measurements were carried out per experimental group.

SEM and FTIR Fourier-transform infrared spectra of as-prepared hydrogel samples were measured using a Varian 640-IR spectrometer equipped with diamond attenuated total reflectance (ATR) optic. Scanning electron microscopy (SEM) images were taken using an Axia Chemi SEM (Thermo Fisher) at an accelerating voltage of 10 kV. Imaging was performed on uncoated, as-applied (d=1.14 mm), hydrogel samples at low vacuum mode. Hydrogel samples were cut with a scalpel and mounted on a 90° angled SEM pin stub using carbon tape.

### Histology

Fresh fallopian tubes (FT) from SBZ Schlachtbetrieb Zurich AG were collected early in the morning, transported on ice, and dissected within 1 h. Fresh tubes were blocked with hydrogel particles, incubated in SOF for 4 h and fixed in 4% Formalin right after incubation to preserve the tissue as well as possible. Fixed tissue samples were sent to Sophilstolab AG, Muttenz, Switzerland for histological analysis. Samples were embedded in paraffin blocks, sliced, and stained using H&E. Images were scanned and analysed using ImageScope.

Biocompatibility of the stimuli-responsive hydrogels throughout the entire procedure associated with their placement, was assessed using a lactate dehydrogenase (LDH) release assay of fibro-blast cells incubated with hydrogel conditioned media and histology of fresh porcine fallopian tubes (FIG. 2). Cytotoxicity of the as-applied (1st Extraction), swollen (2nd Extraction), and degraded (Deg.) hydrogels was investigated by immersion of hydrogels in (2 mL) cell culture media and subsequent cell culture of fibroblasts with the conditioned media (FIG. 2a). The first extraction represents toxicity of hydrogels immediately after placement in the fallopian tubes and the second extraction characterizes the cytotoxic effect of hydrogels after full hydration. Furthermore, to investigate possible cytotoxic effect of degradation products, hydrogels were degraded in media after reaching equilibrium swelling. Lactate dehydrogenase (LDH) re-lease from fibroblasts was measured at different culture durations after incubation with conditioned media (t=4 h, 24 h, 48 h). The data shows negligible cytotoxicity of PL-, TD-Gels and their degradation products and an LDH release comparable to that of fibroblasts cultured in fresh, unconditioned cell medium. Additionally, cell viability using a metabolic activity assay (CellTiterGlo) was assessed after 24 h of incubation with conditioned media and observed to be similar to control cells (FIG. 2b).

To assess tissue compatibility of the implantation and expansion of PL- and TD-Gels, histological analysis of non-blocked and blocked porcine fallopian tubes was performed after in vivo implantation for 3 weeks in a piglet model. Successful placement of the hydrogel was achieved using a small incision in the fallopian tube (FIG. 2c). After three weeks in vivo the hydrogel was identified and histological analysis completed (FIG. 2d). There, the main tissue layer of concern is the inner mucosa, which is composed of ciliated and non-ciliated secretory epithelial cells responsible for tubal fluid production and gamete transport through the fallopian tubes. Functional impairment of the inner lining of the fallopian tubes may result in infertility and ectopic pregnancies. The longitudinal folds of the mucosal tissue (plicae) are clearly visible in the histological H&E stained fallopian tube sections (FIGS. 2d, 2e). Note that the various processing steps of the occluded fallopian tube sections have caused the hydrogel to shrink slightly. However, no apparent damage or deformation of the plicae is visible in the occluded tubes when compared to non-blocked (patent) tubes. In addition, no rips or significant distortions of the smooth muscle layer surrounding the mucosa were observed. The hydrogel showed good contact with fallopian tube and no observable signs of degradation. Additionally, according to the blinded analysis by a certified pathologist, there was no histological difference between the controls and the sample with the implant except some accumulation of fluid and macrophages in the lumen. Importantly, no histological findings for a foreign body reaction could be observed. Together, these results demonstrate that the hydrogels can be placed within the fallopian tube and maintained at the site of interest over several weeks indicating their potential for tube occlusion.

### Endometriosis Model

### Cell Culture

The well characterized, lesion-forming human endometrial cell line EE12Z was purchased from Abm (Applied Biological Materials Inc., USA) after signing a Material Transfer Agreement (MTA). The cell line was maintained according to propagation requirements of manufacturer. Prigrow III medium (Abm TM003) was supplemented with 10% fetal bovine serum (Invitrogen F9665) and 1% of Penicillin/Streptomycin Solution (Gibco P4458) to obtain a complete growth medium. PriCoat™ T25 Flasks (abm, G299) were used for cell culture and media was changed every 2-3 days. Cells were cultured in a humidified incubator (37° C. and 5% CO2) using PriCoat™ T25 Flasks (abm, G299) and cell culture medium was changed every 2-3 days.

### Flow System

To mimic retrograde menstruation through the fallopian tubes, a flow system similar to the burst pressure system was used. A 10 mL syringe (Omnifix® B Braun) was mounted on a programmable syringe pump (NE-1000, KF Technology). A single silicone tube (ID=3 mm) was used to connect the 10 mL syringe with a blunt (20 G) syringe needle. The flow rate and dispensing volume of the programmable syringe pump were adjusted to simulate physiological pressures (bursts of up to 50 mmHg) occurring in the fallopian tube. More precisely, the pump was programmed to enter a continuous loop of dispensing (+0.15 mL) and filling (−0.15 mL) cycles at a rate of 1 mL/min with 10s breaks in between. Evaluation of volumes and flow rates needed to reach physiological pressures were done using the tubing system and the pressure sensor of the burst pressure set-up.

### Endometriosis Model Experiments

Three different sets of experiments were conducted. First, cytocompatibility of the designed flow system was investigated in relevant timeframes. Second, passage of endometrial cells through non-blocked fallopian tube sections were assessed. Third, functional blockage of stimuli degradable hydrogels was evaluated.

To investigate cytocompatibility, the system was filled with 4 mL of cell medium containing 1 million endometrial cells (12Z). A haemostat was used to attach a short silicone tube (ID=1 mm) to the syringe needle of the flow system. To simulate blockage, a second haemostat was used to clamp the short silicone tube. Endometrial cells were subjected to 30 min of continuous dispensing and filling cycles (freq. ˜1.6 cycles/min). Afterwards, cells and media were collected and an LDH cytotoxicity assay was performed immediately (data not shown) and after 24 h of subsequent cell culture. Assessment of endometrial cell passage was done similarly with the difference that either a non-blocked (n=3) or a PL blocked (n=3) fallopian tube segment was attached to the needle. Additionally, the fallopian tube segment was immersed in 4 mL fresh cell media (“Collection Media”). After the experiment, the collection media was centrifuged at 200 g for 2 min and 2 mL of supernatant was carefully removed. Next, cells were carefully resuspended in the remaining 2 mL of collection media and the number of cells which passed though the tubes were counted using a Neubaur chamber and trypan blue staining. Remaining collection media was cultured in 24 well plates at a cell density of 40.000 cells/500 μL and incubated for 24 h. A static control was used as reference. Microscopy images were taken the next day. The experiment was repeated also for durations of 4 hours of continuous dispensing and filling cycles, this time using fixed cells.

### Sperm Passage Experiments

Live boar semen was provided by a fertilization supplier within 2 h of collection. The sperm were counted using a hemocytometer and diluted to 19.6 million cells per mL, similar to the concentration utilized for artificial insemination, using semen extender. The diluted semen sample was loaded into a syringe (4 mL sample volume) and perfusion experiments were conducted for 30 min as described above. After 30 min, the collection media was centrifuged at 200 g for 2 min, the supernatant removed, and the sperm carefully resuspended in 1 mL of semen extender. The number of sperm that passed through the tubes were counted using a hemocytometer and imaged (Zeiss Imager.M2m).

1. Gel comprising a crosslinked network of at least
a) a first monomer unit selected from the group consisting of acrylamide-2-methylpropanesulfonic acid (AMPS), acrylic acid, sodium acrylate, polyethylene glycol acrylate, (acrylamidopropyl)trimethylammonium chloride, methacrylic acid, 2-(dimethylamino)ethyl acrylate, 2-aminoethyl acrylate hydrochloride and 2-acryloxyethyltrimethylammonium chloride or a mixture thereof,
b) a second monomer unit selected from the group consisting of N-2-hydroxyethyl acrylamide (NHEA), N-2-hydroxypropyl acrylamide (NHPA), acrylamide, N-isopropylacrylamide (NIPAm), hydroxyethyl methacrylate (HEMA), hydroxyethyl acrylate (HEA), methyl acrylate, butyl acrylate, ethyl hexyl acrylate, methacrylamide, and ethyl hexyl (meth)acrylate or a mixture thereof;
c) at least one crosslinker with dual functionality, having at least two crosslinking groups and at least one degradable moiety selected from the group consisting of a photo-labile group, a disulfide bond and a diselenide bond.

2. Gel according to claim 1, wherein the crosslinker is a photolabile crosslinker.

3. Gel according to claim 1, wherein the crosslinker comprises a disulfide bond or a diselenide bond.

4. Gel according to claim 1, wherein the first monomer unit is acrylamide-2-methylpropanesulfonic acid (AMPS).

5. Gel according to claim 1, wherein the second monomer unit is N-hydroxyethyl acrylamide (NHEA).

6. Gel according to claim 1 wherein the first monomer unit is acrylamide-2-methylpropanesulfonic acid (AMPS), the second monomer unit is N-hydroxyethyl acrylamide (NHEA) and the crosslinker is either PEGdiPDA or N,N′-bis(acryloyl)cystamine.

7. Gel according to claim 1, wherein the crosslinked network has a combined weight percentage of 20 to 60% by weight, preferably 25% to 40% by weight.

8. Gel according to claim 1, shaped into a tubular form.

9. Gel according to claim 1 for use in the treatment or prevention of endometriosis.

10. Gel according to claim 1 for use as a contraceptive for female individuals.

11. Hydrogel according to claim 1 having a relative swelling ratio of 2 to 20.

12. Hydrogel according to claim 11, shaped into a tubular form.

13. Hydrogel according to claim 11 for use in the treatment or prevention of endometriosis.

14. Hydrogel according to claim 11 for use as a contraceptive for female individuals.

15. Xerogel made of a hydrogel according to claim 11 having a water content of less than 20%, preferably less than 5%.

16. Xerogel according to claim 15, shaped into a tubular form.

17. Xerogel according to claim 15 for use in the treatment or prevention of endometriosis.

18. Xerogel according to claim 15 for use as a contraceptive for female individuals.

19. Aerogel made of a hydrogel according to claim 11 lyophilized via water sublimation or following supercritical drying.

20. Aerogel according to claim 19, shaped into a tubular form.

21. Aerogel according to claim 19 for use in the treatment or prevention of endometriosis.

22. Aerogel according to claim 19 for use as a contraceptive for female individuals.

23. A method of preventing or treating of endometriosis comprising the step of administering gel according to claim 1.

24. A method of contraception comprising administering the gel of claim 1 to a subject in a manner effective to provide contraception.

25. A method of preventing or treating of endometriosis comprising the step of administering a hydrogel according to claim 11.

26. A method of preventing or treating of endometriosis comprising the step of administering a xerogel according to claim 15.

27. A method of preventing or treating of endometriosis comprising the step of administering an aerogel according to claim 19.

28. A method of contraception comprising administering a hydrogel according to claim 11 to a subject in a manner effective to provide contraception.

29. A method of contraception comprising administering a xerogel of claim 15 to a subject in a manner effective to provide contraception.

30. A method of contraception comprising administering an aerogel according to claim 19 to a subject in a manner effective to provide contraception.
